(19) United States
(12) Patent Application Publication
(10) Pub. No.: US 2005/0180999 A9
(43) Pub. Date: Aug. 18, 2005

(54) Cosmetic composition comprising interferential particles and a colouring material

(76) Inventors: Xavier Blin, Paris (FR); Nathalie Jager Lezer, Verrieres-Le-Buisson (FR); Jean-Christophe Simon, Tokyo (JP)

Correspondence Address: FINNEGAN, HENDERSON, FARABOW, GARRETT & DUNNER;LLP, 901 NEW YORK AVENUE, NW, WASHINGTON, DC, 20001-4413, US

(21) Appl. No.: 10/432,329

(22) PCT Filed: Nov. 22, 2001
(86) PCT No.: PCT/FR01/03695

(30) Foreign Application Priority Data
Nov. 23, 2000 (FR) 00/15135

(51) Int. Cl. A61K 7/021; A61K 7/00

(52) U.S. Cl. 424/401; 424/63

(57) ABSTRACT

The subject of the invention is a composition comprising, in a physiologically acceptable medium, interferential particles exhibiting a colour effect and an additional colouring agent, the said colouring agent being present in a sufficient quantity not to mask the colour effect of the said interferential particles. The subject of the invention is also a method for applying make-up to the keratin fibres, and a make-up kit comprising the composition.

The present invention relates to a cosmetic composition containing interferential particles, and more especially to a make-up composition for the skin both of the face and of the human body, of the lips and of superficial body growths such as the nails, the eyelashes, the eyebrows or the hair.

The composition according to the invention may be a coloured make-up composition such as products for the complexion (foundations), make-up for the cheeks or eye shadows, lip products, concealers, blushers, mascaras, eyeliners, make-up products for the eyebrows, lip or eye pencils, nail products, make-up products for the body, make-up products for the hair (hair mascara or lacquer). The composition may be used as it is for application to the keratinous materials or may be applied over a make-up already deposited on the keratinous materials, for example for modifying the make-up (the composition is applied as a top product commonly called top coat).

The make-up composition may also be applied over make-up accessories (support) such as false nails, false eyelashes, pastiches, wigs or over pastilles or patches adhering to the skin or the lips (of the beauty-spot type).

It is known to use fibres in make-up products, in particular for their lengthening effects in mascaras (see JP-A-57/158714), their moisturizing properties in lipsticks (see the document U.S. Pat. No. 5,498,407), for improving the contours of lipsticks on the edges of the lips (see the document EP-A-0106762) or for repairing broken nails (see FR-A-1529329) or in care products for the skin for their velvety feel (JP-A-7/196440). The make-up compositions containing fibres also contain colouring matter such as inorganic or organic pigments.

Fibres exhibiting a colour effect through optical interference, called interferential fibres, are also known from the document EP-A-921217.

However, make-up compositions, such as nail varnishes, are often coloured using colouring matter such as soluble colorants, pigments which are generally metal oxides, such as iron oxides, or using pearlescent agents such as micas coated with metal oxides such as titanium oxide.

However, the inventors have observed that the abovementioned interferential particles can lose their particular colour effect when they are incorporated into make-up compositions containing colouring agents and in particular pigments; in particular, the inventors have observed that if the composition contained an excessive amount of pigments, the latter will mask the particular colour effect of the interferential particles.

The aim of the present invention is therefore to provide a make-up composition not exhibiting the above disadvantages and exhibiting a novel colour effect.

The inventors have discovered that such a composition may be obtained by combining interferential particles with an additional colouring agent in specific quantities. The composition makes it possible to obtain a make-up having the colour effects of interferential particles.

The composition applied to the skin makes it possible to obtain good concealing of skin imperfections and a make-up which illuminates the complexion. The make-up obtained also exhibits a bright visual effect.

Furthermore, the inventors have also discovered that when the interferential particles are interferential fibres, these fibres are very easily incorporated into the cosmetic compositions and become homogeneously distributed in the composition.

The incorporation of interferential fibres into the composition can be done very easily, both in the cold state and in the hot state, without losing the cosmetic and/or optical properties of the composition. In particular, the good homogeneity of the fibres in the composition does not change the appearance of the product. It is possible to incorporate large amounts of fibres into the composition without modifying the visual appearance of the composition.

The composition applied to the keratinous materials also has as an advantage the forming of a deposit exhibiting a velvety feel to the touch due to the homogeneous dispersion of the fibres in the composition and in the deposit formed after the application. The composition therefore provides a different feel to the smooth, crackled or granular feel, thus satisfying consumers seeking novelty.

In addition, the interferential fibres provide properties of mechanical strengthening of the composition and of the deposit formed after application to the keratinous materials, in particular when the deposit comprises a film-forming polymer. In particular, the composition forms a deposit exhibiting good properties of mechanical resistance: the deposit is very resistant to rubbing, to shock and to scratching. The deposit is also very resistant to water (in particular during bathing or showering), rain, tears, sweat and sebum. The fibres thus provide a better retention of the deposit on the keratinous materials.

More precisely, the subject of the invention is a composition comprising, in a cosmetically acceptable medium, interferential fibres exhibiting a colour effect and an additional colouring matter, the said colouring matter being in a sufficient quantity not to mask the colour effect of the said particles.

The subject of the invention is also a cosmetic method for applying make-up to the keratinous materials comprising the application, to the keratinous materials, of a composition as defined above.

The subject of the invention is also the use of interferential particles exhibiting a colour effect and of an additional colouring agent, the said colouring agent being in a sufficient quantity not to mask the colour effect of the said particles, in a composition comprising a physiologically acceptable medium, to obtain a make-up offering high coverage and exhibiting a colour effect through optical interference.

The subject of the invention is also a cosmetic method for applying make-up to the keratinous materials comprising the application to the keratinous materials of a first layer, also called base layer, of a first cosmetic composition comprising, in a cosmetically acceptable medium, at least one colouring agent, then the application, to at least a portion of the said first layer, of a second layer of a second cosmetic composition comprising, in a cosmetically acceptable medium, interferential particles and a second additional colouring agent, the said colouring agent being in a sufficient quantity not to mask the colour effect of the said particles, the first composition not comprising interferential particles as present in the second composition.

The subject of the invention is also a make-up kit comprising:
a first composition comprising, in a cosmetically acceptable medium, a first colouring agent, and
a second composition comprising, in a cosmetically acceptable medium, interferential particles and a second additional colouring agent, the said colouring agent being in a sufficient quantity not to mask the colour effect of the said particles,
   the first composition not comprising interferential particles as present in the second composition, the first and second compositions being packaged in separate containers.

The subject of the invention is also a support to which make-up has been applied, such as the make-up accessories cited above, comprising a make-up which is capable of being obtained according to the make-up application method as defined above and applied to the said support.

As the interferential particles confer a novel visual effect, when they are formulated in a transparent or translucent carrier, the composition may be applied as a top coat over a make-up already deposited on keratinous materials so as to thus modify the appearance of the make-up. In addition, the particular colour effect of the make-up is clearly visible when the composition is applied to dark skins such as ethnic skins.

In the present application, “physiologically acceptable medium” is understood to mean a medium compatible with the keratinous materials of human beings such as the skin, nails, hair, eyelashes, eyebrows, as a cosmetic medium.

The interferential particles may be organic particles or inorganic particles.

In a first embodiment of the composition according to the invention, the interferential particles may be interferential fibres.

The expression “fibre” should be understood to mean an object having a length L and a diameter D such that L is much greater than D, D being the diameter of the circle in which the section of the fibre is inscribed. In particular, the ratio L/D (or shape factor) is chosen from the range going from 1.2 to 2500, preferably from 1.5 to 500, and better still from 1.6 to 150.

The fibres which can be used in the composition of the invention may be fibres of synthetic or organic origin, and more particularly fibres of a synthetic polymer.

In particular, the interferential fibres may be fibres with a multilayer structure of polymer, the said layers being such that they allow the creation of a colour effect by interferences of light rays, which diffract and scatter differently according to the layers. Thus, such fibres may have colours which vary according to the angle of observation and the incidence of light, and may confer iridescent glints. Fibres with multilayer structure of polymers are in particular described in the documents EP-A-921217, EP-A-686858 and U.S. Pat. No. 5,472,798.

The multilayer structure may comprise at least two layers, each layer, independently or otherwise of the other layer(s), being made of at least one synthetic polymer.

The fibre with multilayer structure preferably has a reflection spectrum such that the width at half height of the spectrum λ_{L=1/2} is in the range 0<λ_{L=1/2<}200 nm.

The interferential fibre may be formed of alternate individual layers of polymers having different refractive indices; each layer being in a plane (P) parallel to the direction of the principal axis of the fibre, in the direction of its length L. According to the thickness of each of the different layers, different colours are obtained. In general, the structure is composed of alternating layers of low refractive index and of high refractive index. Thus, in a cross section to the direction of the axis of the length L of the fibre, the fibre has a multilayer structure comprising alternate layers of at least a first polymer and a second polymer.

The multilayer part of the fibre may comprise at least 5 individual layers of polymer, in particular from 5 to 120, preferably at least 10 layers, in particular from 10 to 70 layers, and better still from 10 to 50 layers.

Each layer of the first and second polymers has respectively a thickness d₁, d₂ which may range, independently of each other, from 0.02 μm to 0.3 μm, and preferably from 0.05 μm to 0.15 μm.

Advantageously, the polymers present in the fibres advantageously have a refractive index ranging from 1.30 to 1.82 and better still ranging from 1.35 to 1.75. In particular, the first and second polymers have respectively a refractive index n₁ and n₂ such that n₁/n₂ ranges from 1.1 to 1.4.

Advantageously, n₁, n₂, d₁, d₂ satisfy the equation:
λ=2(n₁d₁+n₂d₂)=2n₁[d₁+d₂(n₂/n₁)]
in which λ is the wavelength, expressed in μm, of the colour of the fibre formed by optical interference (wavelength of the peak of the reflection spectrum); d₁ and d₂ being expressed in μm.

The fibres which can be used in the composition according to the invention are preferably polyester, acrylic polymer and polyamide fibres.

The polymers constituting the fibres which are particularly preferred are polyesters such as polyethylene terephthalate, polyethylene naphthalate, polycarbonate; acrylic polymers such as polymethyl methacrylate; polyamides.

The polyethylene terephthalate may be obtained by polycondensation of terephthalic acid with ethylene glycol. It may contain from 0 to 30% by weight, preferably from 0 to 15% by weight, and better still from 0 to 10% by weight, relative to the total weight of the monomers, of other comonomers.

In particular, the polyethylene terephthalate may comprise from 0.3 to 10 mol %, and preferably from 0.5 to 5 mol %, of dicarboxylic acid monomer comprising a sulphonic acid group neutralized by a metal salt, relative to the total weight of the dicarboxylic acid monomer present in the polyethylene terephthalate.

The sulphonic acid group neutralized by a metal salt is a group of formula —SO₃M in which M is a metal, preferably an alkali or alkaline-earth metal, and more particularly sodium, potassium or lithium.

As example of dicarboxylic acid monomer comprising a sulphonic acid group neutralized by a metal salt, there may be used the sodium salt of 5-sulphoisophthalic acid, the potassium salt of 5-sulphoisophthalic acid, the lithium salt of 5-sulphoisophthalic acid, and the methyl diesters thereof, sodium 3,5-di(β-hydroxyethoxycarbonyl)benzenesulphonate, potassium 3,5-di(β-hydroxyethoxycarbonyl)benzenesulphonate, lithium 3,5-di(β-hydroxyethoxycarbonyl)benzenesulphonate, sodium 4-sulphonate-2,6-naphthalic acid methyl diester, potassium 4-sulphonate-2,6-naphthalic acid methyl diester, lithium 4-sulphonate-2,6-naphthalic acid methyl diester, sodium 2,6-dicarboxynaphthalene-4-sulphonate, sodium 2,6-dicarboxynaphthalene-1-sulphonate, 3-sulphonate-2,6-naphthalic acid methyl diester, sodium 4,8-disulphonate-2,6-naphthalic acid methyl diester, sodium 2,6-dicarboxynaphthalene-4,8-disulphonate, sodium 2,5-bis(hydroxyethoxy)benzenesulphonate, sodium sulphosuccinate, and mixtures thereof. Sodium 5-sulphoisophthalic acid methyl diester, the sodium salt of 5-sulphoisophthalic acid and sodium 3,5-di(β-hydroxyethoxycarbonyl)benzenesulphonate are preferably used.

The polyethylene naphthalate may be obtained by polycondensation of 2,6-naphthalic acid or of 2,7-naphthalic acid with ethylene glycol. The polyethylene naphthalate may therefore be a polyethylene-2,6-naphthalate or a polyethylene-2,7-naphthalate, preferably a polyethylene-2,6-naphthalate.

It may contain from 0.3 to 5 mol % of dicarboxylic acid monomer comprising a sulphonic acid group neutralized by a metal salt as defined above, relative to the total weight of the dicarboxylic acid monomer present in the polyethylene naphthalate.

Other comonomers such as an additional dicarboxylic acid, different from the dicarboxylic acids mentioned above, or an additional diol, different from polyethylene glycol, may be present in the polyethylene terephthalate or the polyethylene naphthalate.

The additional dicarboxylic acid may be chosen from the aromatic dicarboxylic acids such as isophthalic acid, biphenyldicarboxylic acid, 4,4′-dicarboxylic acid of diphenyl ether, 4,4′-dicarboxylic acid of diphenylmethane, 4,4′-dicarboxylic acid of diphenyl sulphone, 4,4′-dicarboxylic acid of 1,2-diphenoxyethane, 2,5-pyridinedicarboxylic acid, 2,6-naphthalenedicarboxylic acid, 2,7-naphthalenedicarboxylic acid, dicarboxylic acid of diphenyl ketone; aliphatic dicarboxylic acids such as malonic acid, succinic acid, adipic acid, azelaic acid, sebacic acid; alicyclic dicarboxylic acids such as dicarboxylic acid of decalin; hydroxycarboxylic acids such as β-hydroxyethoxybenzoic acid, para-hydroxybenzoic acid and hydroxypropionic acid.

The additional diol may be chosen in particular from the aliphatic diols such as propylene glycol, butylene glycol, hexylene glycol, diethylene glycol, polyethylene glycol; the aromatic diols such as hydroquinone, catechol, naphthalenediol, resorcinol, bisphenol A; alicyclic diols such as cyclohexanedimethanol.

Other comonomers may also be chosen from polyvalent carboxylic acids such as trimellitic acid, pyromellitic acid, tricarballylic acid; polyhydric alcohols such as glycerin, trimethylolethane, trimethylolpropane and pentaerythritol.

The polymethyl methacrylate may comprise acid monomers such that the acid value of the polymer is preferably greater than 3, in particular ranging from 3 to 20, and better still from 4 to 15. Such acid monomers may be (meth)acrylic acid or maleic acid.

The polyamide may be chosen from nylon 6, nylon 6-6, nylon 6-12, nylon 11, nylon 12, whose chemical composition is well known to persons skilled in the art.

Advantageously, in the multilayer structure of the flat fibres, the first polymer may be chosen from polyesters such as polyethylene terephthalate, polyethylene naphthalate, polycarbonate, in particular those defined above; the second polymer may be chosen from acrylic polymers such as polymethyl methacrylate, and the polyamides, in particular those described above.

Moreover, the fibres may be surface-treated or otherwise, coated with a protective layer or otherwise.

The fibres with multilayer structure may comprise a protective layer which may comprise a polymer chosen from the layer polymers. Preferably, the polymer of the protective layer may have a refractive index ranging from 1.35 to 1.55.

The thickness of the protective layer may be greater than the thickness of the layers of polymers of the multilayer part.

The thickness of the protective layer may range from 2 μm to 10 μm, preferably from 2 μm to 7 μm.

As polymer of the protective layer, there may be used in particular polytetrafluoroethylene, tetrafluoroethylene/propylene copolymers, tetrafluoroethylene/hexafluoropropylene copolymers, tetrafluoroethylene/ethylene copolymers, tetrafluoroethylene/tetrafluoropropylene copolymers, polyvinylidene fluoride, polypentadecafluorooctyl acrylates, polyfluoroethyl acrylates, polytrifluoroisopropyl methacrylates, polytrifluoroethyl methacrylates, polyethyl acrylates, polyethyl methacrylates. It is also possible to use silicone polymers such as polydimethylsilanes, polydimethylsiloxanes; polyurethanes.

The interferential fibres may be obtained in a known manner by extrusion of the polymer(s) through a die of rectangular shape and then cutting the thread obtained to the desired length.

The fibres may be unitary (or monofilament) or organized, for example plaited (or multifilament). When the fibres are multifilament fibres, each filament may be of a different chemical composition and may have a different colour: multifilament fibres having different colours are thus obtained. In particular, their ends are blunt and/or smooth to avoid injury.

Advantageously, the fibres are insoluble in water.

The fibre may be twisted along the axis of the length L of the fibre. When the fibre is not twisted, it exhibits a colour in a certain angle of view; outside this angle, the fibre is transparent or white in colour. The flat twisted fibre, for its part, exhibits a colour regardless of the angle of observation.

In particular, the fibres have a length ranging from 1 μm to 10 mm, preferably from 0.1 mm to 5 mm and better still from 0.3 mm to 3.5 mm. Their section may be included in a circle having a diameter ranging from 2 nm to 500 μm, preferably ranging from 100 nm to 100 μm and better still from 1 μm to 70 μm.

Advantageously, the fibres have a cross section (section perpendicular to the axis of the direction of the length of the fibre) having a longer length L1 and a shorter length L2 (L2 corresponds to the thickness of the fibre) such that L1/L2 (the ratio L1/L2 is also called flattening factor) is greater than or equal to 4, preferably greater than 7. In particular, L1/L2 ranges from 4 to 15, preferably from 6 to 12, and better still from 7 to 10. Thus, the cross section of the fibre has a flat shape. Advantageously, the longer length L1 and the shorter length L2 define axes X1, X2, respectively, such that the axis X1 is substantially perpendicular to the axis X2. The longer length L1 corresponds to the diameter D of the fibre as mentioned above. The fibres may in particular have a cross section which is substantially rectangular, ovoid or ellipsoid. Thus, the fibres may be provided in the form of ribbons or tagliatelle.

The weight or titre of the fibres is often given in denier or decitex and represents the weight, in grams, per 9 km of thread. Preferably, the fibres according to the invention have a titre chosen from the range going from 0.15 to 30 denier and better still from 0.18 to 18 denier.

As interferential fibres, there may be used the fibres sold under the names “Morphotex” by the company TEIJIN. Such fibres are described in application EP-A-921217 whose content is integrated by way of reference into the present application.

In a second embodiment of the composition according to the invention, the interferential particles may be interferential goniochromatic pigments. These pigments have a distinct particle shape from the interferential fibres described above.

The expression goniochromatic pigment with interferential multilayer structure, called interferential goniochromatic pigment according to the invention, is understood to mean a pigment with an at least bilayer structure, the said layers being such that they allow the creation of a colour effect by interference of the light rays, which diffract and scatter differently according to the layers. Thus, such pigments may have colours which vary according to the angle of observation and the incidence of the light, and may confer iridescent glints.

The multilayer structure may comprise at least two layers, each layer, independently of the other layer(s) or otherwise, being made of at least one material chosen from the group consisting of the following materials: MgF₂, CeF₃, ZnS, ZnSe, Si, SiO₂, Ge, Te, Fe₂O₃, Pt, Va, A1₂O₃, MgO, Y₂O₃, S203, SiO, HfO₂, ZrO₂, CeO₂, Nb₂O₅, Ta₂O₅, TiO₂, Ag, Al, Au, Cu, Rb, Ti, Ta, W, Zn, MoS₂, cryolite, alloys, polymers and combinations thereof.

Preferably, the goniochromatic pigment with interferential interlayer structure according to the invention is chosen from the group consisting of the following commercial goniochromatic pigments: Infinite Colors from SHISEIDO, Sicopearl Fantastico from BASF, Colorstream from MERCK, Colorglitter from 3M and Chromaflair from FLEX.

Consequently, the multilayer structure may be essentially inorganic or organic. Different colours are obtained according to the thickness of each of the different layers.

The pigments with interferential monolayer structure according to the invention are in particular those described in the following documents: U.S. Pat. No. 3,438,796, EP-A-227423, U.S. Pat. No. 5,135,812, EP-A-170439, EP-A-341002, U.S. Pat. No. 4,930,866, U.S. Pat. No. 5,641,719, EP-A-472371, EP-A-395410, EP-A-753545, EP-A-768343, EP-A-571836, EP-A-708154, EP-A-579091, U.S. Pat. No. 5,411,586, U.S. Pat. No. 5,364,467, WO-A-97/39066, DE-A-4 225 031, WO 9517479 (BASF), DE-A-196 14 637 and combinations thereof. They are provided in the form of glitter, with a metallic colour.

For example, the interferential multilayer structure is chosen from the group consisting of the structures: Fe₂O₃/SiO₂/Fe₂O₃/SiO₂/Fe₂O₃; MoS₂/SiO₂/micaoxide/SiO₂/MoS₂; Fe₂O₃/SiO₂/mica-oxide/SiO₂/Fe₂O₃.

In general, the structure is composed of an alternation of layers with a low optical index and a high optical index.

The interferential particles may be provided in the composition according to the invention, or in the top composition, in an amount ranging from 0.01% to 50% by weight, relative to the total weight of the composition, preferably from 0.1% to 30% by weight, and better still from 0.3% to 20% by weight.

The colouring agent present in the composition according to the invention, or in the base and/or top composition, is different from the interferential particles described above. The additional colouring agent may be chosen from pigments, pearlescent agents, colorants and mixtures thereof.

The expression pigments should be understood to mean particles of any form, which are white or coloured, inorganic or organic, insoluble in saline, intended to colour the composition.

The expression pearlescent agents should be understood to mean iridescent particles of any form, in particular which are produced by certain molluscs in their shell, or synthesized.

The pigments may be present in the composition, in particular in the base and/or top composition, in an amount of 0 to 15% (in particular 0.01% to 15%) relative to the weight of the composition, preferably from 0.01% to 10% by weight, and better still from 0.02% to 5% by weight.

The pigments may be white or coloured, inorganic and/or organic. There may be mentioned, among the inorganic pigments, titanium dioxide, optionally surface-treated, zirconium or cerium oxides, and zinc, iron (black, yellow or red) or chromium oxides, manganese violet, ultramarine blue, chromium hydrate and ferric blue, metallic powders such as aluminium powder and copper powder.

Among the organic pigments, there may be mentioned carbon black, D & C type pigments, and lacquers based on carmine, barium, strontium, calcium or aluminium.

The pearlescent agents may be present in the composition, in particular in the base and/or top composition, in an amount of 0 to 25% (in particular 0.01% to 25%) by weight, relative to the total weight of the composition, preferably from 0.01% to 15% by weight, and better still from 0.02% to 5% by weight.

The pearlescent pigments may be chosen from white pearlescent pigments such as mica coated with titanium, or with bismuth oxychloride, coloured pearlescent pigments such as mica-titanium coated with iron oxides, mica-titanium coated with in particular ferric blue or chromium oxide, mica-titanium coated with an organic pigment of the abovementioned type and the pearlescent pigments based on bismuth oxychloride.

The colouring agent may also be a colouring matter chosen from water-soluble or fat-soluble colorants or colouring polymers. The colouring matter may be present in the composition, in particular the base and/or top composition, in an amount of colorant active substance ranging from 0 to 6% (in particular 0.01% to 6%) by weight, relative to the total weight of the composition, preferably ranging from 0.01% to 3% by weight.

The fat-soluble colorants are for example soya bean oil, Sudan brown, DC Yellow 11, DC orange 5, quinoline yellow, Sudan Red III (CTFA name D&C red 17), lutein, quinizarin green (CTFA name DC green 6), Alizurol purple SS (CTFA name DC violet No. 2), carotenoid derivatives such as lycopene, beta-carotene, bixin, capsantein and/or mixtures thereof.

Among the water-soluble colorants, there may be mentioned extracts of dye plants, such as for example Aleurites Moluccana Willd, Alkanna Tinctoria. Tausch, Areca Catechu L., Arrabidaea Chica E. and B., Bixa Orellana L (annatto), Butea Monosperma Lam, Caesalpina Echinata Lam, Caesalpina Sappan L., Calophyllum Inophyllum L., Carthamus Tinctorius L., Cassia Alata L., Chrozophora Tinctoria L., Crocus Sativus L., Curcuma Longa L., Diospyros Gilletii de Wild, Eclipta Prostrata L., Gardenia Erubescens Stapf. and Hutch., Gardenia Terniflora Schum. and Thonn., Genipa Americana L., Genipa Brasiliensis L., Guibourtia Demeusei (Harms) J. Leon, Haematoxylon Campechianum L., Helianthus annuus, Humiria Balsamifera (Aubl.) St-Hil., Isatis Tinctoria L., Mercurialis perenis, Monascus purpureus, Monascus ruber, Monascus pilosus, Morus Nigra L., Picramnia Spruceana, Pterocarpus Erinaceus Poir., Pterocarpus Soyauxii Taub., Rocella Tinctoria L., Rothmannia Whitfieldii (Lindl.) Dand., Schlegelia Violacea (Aubl.) Griseb., Simira Tinctoria Aublet, Stereospermum Kunthianum Cham., Symphonia Globulifera L., Terminalia Catappa L., Sorgho, Aronia melanocarpa, naphthoquinone including lawsone, derived from Lawsonia Inermis L. also called henna or Impatiens Balsamina, extracts of redwood as described in the document WO98/44902, beet juice, disodium salt of fuchsin, anthocyanins such as extracts of red fruit, dihydroxyacetone, mono- or polycarbonyl derivatives such as isatin, alloxan, nihhydrin, glyceraldehyde, mesotartaric aldehyde, 4,5-pyrazolinedione derivatives, and mixtures thereof, it being possible for these skin colouring agents to be combined or otherwise with direct dyes or indole derivatives, and/or mixtures thereof.

These dye plant extracts may be in the form of a lyophilizate, a paste or a solution: generally, the leaves of the dye plant are ground in order to obtain a powder. This powder is placed in an aqueous phase for a few hours. The mixture is then centrifuged and then filtered. The filtrate obtained is frozen and then freeze-dried.

The colouring agent may also be a colouring polymer, that is to say a polymer comprising at least one organic colouring group. The colouring polymer contains in general less than 10% by weight, relative to the total weight of the polymer, of colouring matter.

The colouring polymer may be of any chemical nature, in particular a polyester, polyamide, polyurethane, polyacrylic, poly(meth)acrylic, polycarbonate, polymers of natural origin such as cellulose or chitosan polymers, or mixtures thereof, and preferably polyester or polyurethane polymers.

The colouring polymer may comprise a colouring group, may be grafted, in particular by a covalent bond, onto the polymer chain, as described in the documents WO-A-96/29046, WO-A-92/01022, WO-A-90/07558, BE-A-609054.

In particular, the colouring polymer may be a copolymer based on at least two distinct monomers of which at least one is an organic colouring monomer.

The monomers of the colouring polymer may be chosen from anthraquinones, methines, bismethines, azamethines, arylidenes, 3H-dibenzo[7,i-j]isoquinolines, 2,5-diarylaminoterephthalic acids and their esters, phthaloylphenothiazines, phthaloylphenoxazines, phthaloylacridone, anthrapyrimidines, anthrapyrazoles, phthalocyanins, quinophthalones, indophenols, perinones, nitroarylamines, benzodifuran, 2H-1-benzopyran-2-one, quinophthalones, perylenes, quinacridones, triphenodioxazines, fluoridines, 4-amino-1,8-naphthalimides, thioxanthrones, benzanthrones, indanthrones, indigo, thioindigo, xanthene, acridine, azine, oxazine.

Colouring monomers are in particular described in the documents U.S. Pat. No. 4,267,306; U.S. Pat. No. 4,359,570; U.S. Pat. No. 4,403,092; U.S. Pat. No. 4,617,373; U.S. Pat. No. 4,080,355; U.S. Pat. No. 4,740,581; U.S. Pat. No. 4,116,923; U.S. Pat. No. 4,745,173; U.S. Pat. No. 4,804,719; U.S. Pat. No. 5,194,463; U.S. Pat. No. 5,804,719; WO-A-92/07913.

Polymeric colorants are in particular described in the documents U.S. Pat. No. 4,804,719; U.S. Pat. No. 5,032,670; U.S. Pat. No. 4,999,418; U.S. Pat. No. 5,106,942; U.S. Pat. No. 5,030,708; U.S. Pat. No. 5,102,980; U.S. Pat. No. 5,043,376; U.S. Pat. No. 5,194,463; WO-A-92/07913; WO-A-97/24102, whose content is incorporated by way of reference into the present application.

Sulphopolyester colouring polymers such as those described in the document WO-A-97/24102 are preferably used.

The colouring polymers may be present in the composition according to the invention, in particular in the base and/or top composition, in an amount ranging from 0% to 50% by weight (0.01% to 50%), relative to the total weight of the composition, preferably ranging from 0.5% to 25% by weight, and better still from 0.2% to 20% by weight.

Advantageously, the interferential particles and the additional colouring agent may be present in the composition according to the invention, or in the top composition, in a weight ratio interferential particles/active substance of the additional colouring agent greater than or equal to 2 (in particular ranging from 2 to 500), and better still greater than or equal to 5 (in particular ranging from 5 to 500).

In addition to the additional colouring agent, the composition according to the invention may contain, in addition, fillers. The expression fillers should be understood to mean particles of any form, which are colourless or white, inorganic or synthetic, insoluble in the medium of the composition regardless of the temperature at which the composition is produced. These fillers also serve to modify the rheology or the texture of the composition.

The fillers may be inorganic or organic, of any form, platelet, spherical or oblong, regardless of the crystallographic form (for example sheet, cubic, hexagonal, orthorhombic and the like). There may be mentioned talc, mica, silica, kaolin, powders of polyamide (Nylon®) (Orgasol® from Atochem), of poly-β-alanine and of polyethylene, powders of tetrafluoroethylene polymers (Teflon®), lauroyllysine, starch, boron nitride, polymeric hollow microspheres such as those of polyvinylidene chloride/acrylonitrile such as Expancel® (Nobel Industrie), acrylic acid copolymers (Polytrap® from the company Dow Corning) and microbeads of silicone resin (Tospearls from Toshiba, for example), particles of elastomeric polyorganosiloxanes, precipitated calcium carbonate, magnesium carbonate and hydrocarbonate, hydroxyapatite, hollow microspheres of silica (Silica Beads® from Maprecos), glass or ceramic microcapsules, metal soaps derived from organic carboxylic acids having from 8 to 22 carbon atoms, preferably from 12 to 18 carbon atoms, for example zinc, magnesium or lithium stearate, zinc laurate, magnesium myristate.

The fillers may be present in an amount of 0 to 90% by weight, relative to the total weight of the composition, in particular of the base and/or top composition, preferably 0.01 to 50% by weight, and better still from 0.02% to 30% by weight.

The composition of the invention, or the base and/or top composition, may comprise a particulate phase comprising the pigments and/or the pearlescent agents and/or the fillers as described above, which may be present in an amount of 0 to 98% (in particular 0.01% to 98%) of the total weight of the composition, preferably from 0.01% to 30%, and better still from 0.02% to 20%.

The composition according to the invention, or one of the base and/or top compositions, may comprise a hydrophilic cosmetic medium or a lipophilic medium.

The composition, or one of the base and/or top compositions, may comprise water or a mixture of water and hydrophilic organic solvents such as alcohols and in particular linear or branched lower monoalcohols having from 2 to 5 carbon atoms such as ethanol, isopropanol or n-propanol, polyols such as glycerin, diglycerin, propylene glycol, sorbitol, pentylene glycol, polyethylene glycols. The hydrophilic phase may, in addition, contain C₂ ethers and C₂-C₄ aldehydes which are hydrophilic. The water or the mixture of water and hydrophilic organic solvents may be present in the composition according to the invention, or one of the base and/or top compositions, in an amount ranging from 0% to 90% (in particular 0.1% to 90%) by weight, relative to the total weight of the composition, and preferably from 0% to 60% by weight (in particular 0.1% to 60% by weight).

The composition, or one of the base and/or top compositions, may also comprise a fatty phase, in particular consisting of fatty substances which are liquid at room temperature (25° C. in general) and/or fatty substances which are solid at room temperature such as waxes, pasty fatty substances, gums and mixtures thereof. This fatty phase may, in addition, contain lipophilic organic solvents.

As fatty substances which are liquid at room temperature, often called oils, which can be used in the invention, there may be mentioned: hydrocarbon oils of animal origin such as perhydrosqualene; vegetable hydrocarbon oils such as liquid triglycerides of fatty acids with 4 to 10 carbon atoms such as the triglycerides of heptanoic or octanoic acids, or sunflower, maize, soyabean, grapeseed, sesame, apricot, macadamia, castor and avocado oils, the triglycerides of caprylic/capric acids, jojoba oil, shea butter; linear or branched hydrocarbons, of mineral or synthetic origin, such as paraffin oils and derivatives thereof, petroleum jelly, polydecenes, hydrogenated polyisobutene such as parleam; synthetic esters and ethers in particular of fatty acids, such as for example Purcellin oil, isopropyl myristate, 2-ethylhexyl palmitate, 2-octyldodecyl stearate, 2-octyldodecyl erucate, isostearyl isostearate; hydroxylated esters such as isostearyl lactate, octyl hydroxystearate, octyldodecyl hydroxystearate, diisostearyl malate, triisocetyl citrate, heptanoates, octanoates, decanoates of fatty alcohols; polyol esters such as propylene glycol dioctanoate, neopentyl glycol diheptanoate, diethylene glycol diisononanoate; and pentaerythritol esters; fatty alcohols having from 12 to 26 carbon atoms such as octyl dodecanol, 2-butyloctanol, 2-hexyldecanol, 2-undecylpentadecanol, oleyl alcohol; partially hydrocarbon- and/or silicone-based fluorinated oils; silicone oils such as polymethylsiloxanes (PDMS) which are volatile or otherwise, linear or cyclic, liquid or pasty at room temperature such as cyclomethicones, dimethicones, optionally comprising a phenyl group, such as phenyl trimethicones, phenyltrimethylsiloxydiphenylsiloxanes, diphenylmethyldimethyltrisiloxanes, diphenyl dimethicones, phenyl dimethicones, polymethylphenylsiloxanes; mixtures thereof.

These oils may be present in an amount ranging from 0.01 to 90%, and better still from 0.1 to 85% by weight, relative to the total weight of the composition.

The composition, or one of the base and/or top compositions, according to the invention may also comprise one or more cosmetically acceptable organic solvents (acceptable tolerance, toxicology and feel). These solvents may be present in an amount ranging from 0 to 90%, and better still from 0 to 60% by weight, relative to the total weight of the composition, and better still from 0.1 to 30%.

As solvents which can be used in the composition of the invention, there may be mentioned acetic acid esters such as methyl, ethyl, butyl, amyl, 2-methoxyethyl or isopropyl acetate; ketones such as methyl ethyl ketone, methyl isobutyl ketone; hydrocarbons such as toluene, xylene, hexane, heptane; aldehydes having from 5 to 10 carbon atoms; ethers having at least 3 carbon atoms; and mixtures thereof.

The composition of the invention, or one of the base and/or top compositions, may in addition advantageously comprise a fatty substance which is solid or pasty at room temperature, such as gums or waxes. The waxes may be hydrocarbon-based, fluorinated and/or silicone-based and may be of plant, mineral, animal and/or synthetic origin. In particular, the waxes have a melting point greater than 25° C. and better still greater than 45° C.

As wax which can be used in the composition of the invention, there may be mentioned beeswax, Carnauba or Candelilla wax, paraffin, microcrystalline waxes, ceresine or ozokerite; synthetic waxes such as polyethylene or Fischer-Tropsch waxes, silicone waxes such as alkyl or alkoxy dimethicone having from 16 to 45 carbon atoms.

The gums are generally polydimethylsiloxanes (PDMS) of high molecular weight or cellulose gums or polysaccharides and the pasty substances are generally hydrocarbon compounds such as lanolins and derivatives thereof or PDMSs.

The nature and the quantity of the solid substances depend on the desired mechanical properties and textures. As a guide, the composition may contain from 0 to 50% by weight of waxes, relative to the total weight of the composition, and better still from 1 to 30% by weight.

The composition according to the invention, or one of the base and/or top compositions, may in addition comprise a film-forming polymer. In the present application, the expression “film-forming polymer” is understood to mean a polymer capable of forming, on its own or in the presence of a film-forming aid, a continuous and adherent film on a support, in particular on keratinous material.

The film-forming polymer may be chosen from vinyl polymers, polycondensates or polymers or natural origin. As film-forming polymer, there may be mentioned in particular acrylic polymers, polyurethanes, polyesters, polyamides, polyureas, cellulose polymers. The film-forming polymer may be dissolved or dispersed in the form of solid particles in the physiologically acceptable medium of the composition.

The film-forming polymer may be present in the composition according to the invention, or one of the base and/or top compositions, in a polymer dry matter content ranging from 0.01% to 60% by weight relative to the total weight of the composition, preferably from 0.5% to 40% by weight, and better still from 1% to 30% by weight.

The film-forming polymer may be combined with film-forming aids. Such a film-forming agent may be chosen from all the compounds known to persons skilled in the art to be capable of fulfilling the desired function, and may be chosen in particular from plasticizing agents and coalescing agents.

The composition according to the invention, or one of the base and/or top compositions, may be provided in particular in the form of a suspension, dispersion, solution, gel, emulsion, in particular oil-in-water (O/W) or water-in-oil (W/O), or multiple (W/O/W or polyol/O/W or O/W/O) emulsion, in the form of a cream, paste, foam, dispersion of vesicles in particular of ionic or nonionic lipids, two-phase or multiphase lotion, spray, powder, paste, in particular soft paste (in particular paste having a dynamic viscosity at 25° C. of the order of 0.1 to 40 Pa.s at a shear rate of 200 s⁻¹, after 10 minutes of measurement in a cone/planar geometry). The composition may contain an organic, in particular anhydrous, continuous phase.

Persons skilled in the art will be able to choose the appropriate galenic form, as well as its method of preparation, on the basis of their general knowledge, taking into account, on the one hand, the nature of the constituents used, in particular their solubility in the carrier, and, on the other hand, the application envisaged for the composition.

The composition according to the invention, or one of the base and/or top compositions, may also contain ingredients commonly used in cosmetics, such as vitamins, thickeners (in particular optionally modified clays), trace elements, emollients, sequestrants, perfumes, alkalinizing or acidifying agents, preservatives, UV-screening agents, or mixtures thereof.

Of course, persons skilled in the art will be careful to choose this or these optional additional compounds, and/or their quantity, so that the advantageous properties of the composition according to the invention are not, or not substantially, impaired by the addition envisaged.

The composition of the invention, in particular the base and top compositions, may be obtained according to the methods of preparation conventionally used in the cosmetics or dermatological fields.

The examples of compositions below are given by way of illustration and without limitation.

### EXAMPLE 1

A loose face powder was prepared comprising:

| | | |
|---|---|---|
| Nylon-12 powder | 30 | g |
| Interferential fibres of polyethylene | 10 | g |
| terephthalate and of nylon having a length of | | |
| 0.3 mm sold under the name “Morphotex” by the | | |
| company TEIJIN | | |
| Iron oxides | 3.5 | g |
| Silicone binder | 3 | g |
| Talc qs | 100 | g |

The powder confers a radiant make-up when applied to the face.

### EXAMPLE 2

A foundation having the following composition was prepared:

| | | |
|---|---|---|
| Cetyl dimethicone copolyol/polyglyceryl | 0.5 | g |
| 4-isostearate/hexyl laurate mixture sold under | | |
| the trade name “Abil WE 09” by the company | | |
| Goldschmidt | | |
| Interferential fibres of polyethylene | 10 | g |
| terephthalate and of nylon having a length of | | |
| 0.3 mm sold under the name “Morphotex” by the | | |
| company TEIJIN | | |
| Iron oxides | 0.5 | g |
| Volatile silicone (DC245 Fluid from the | 15 | g |
| company Dow Corning) | | |
| Water qs | 100 | g |

The foundation confers a radiant make-up on the complexion when applied to the face.

### EXAMPLE 3

A mascara having the following composition was prepared:

| | | |
|---|---|---|
| Carboxymethyl cellulose | 15 | g |
| Laponite | 0.2 | g |
| Interferential fibres of polyethylene | 10 | g |
| terephthalate and of nylon having a length of | | |
| 0.3 mm sold under the name “Morphotex” by the | | |
| company TEIJIN | | |
| Disodium salt of fuchsin | 0.05 | g |
| Water qs | 100 | g |

The eyelashes to which this mascara has been applied exhibit a novel colour effect.

### EXAMPLE 4

A nail varnish having the following composition was prepared:

| | | |
|---|---|---|
| Nitrocellulose | 17.1 | g |
| N-Ethyl-o,p-toluenesulphonamide | 5.4 | g |
| Tributyl acetyl citrate | 5.4 | g |
| Interferential fibres of polyethylene | 10 | g |
| terephthalate and of nylon having a length of | | |
| 0.3 mm sold under the name “Morphotex” by the | | |
| company TEIJIN | | |
| DC Red 34 | 0.025 | g |
| Hectorite | 1.0 | g |
| Isopropyl alcohol | 7.2 | g |
| Ethyl acetate, butyl acetate qs | 100 | g |

This nail varnish may be applied directly to the nails or to a base layer obtained after application of a nail varnish base having the following composition:

| | | |
|---|---|---|
| Nitrocellulose | 19 | g |
| N-Ethyl-o,p-toluenesulphonamide | 6 | g |
| Tributyl acetyl citrate | 6 | g |
| Midnight blue pigments | 1 | g |
| Hectorite | 1.2 | g |
| Isopropyl alcohol | 8 | g |
| Ethyl acetate, butyl acetate qs | 100 | g |

A make-up is obtained on the nails exhibiting a colour effect on a midnight blue base.

1. Composition comprising, in a physiologically acceptable medium, interferential particles exhibiting a colour effect and an additional colouring agent, the said colouring agent being present in a sufficient quantity not to mask the colour effect of the said interferential particles.

2. Composition according to claim 1, characterized in that the interferential particles are organic particles.

3. Composition according to claim 1 or 2, characterized in that the interferential particles are fibres.

4. Composition according to claim 3, characterized in that the interferential fibres are synthetic polymer fibres.

5. Composition according to claim 3 or 4, characterized in that the interferential fibres are chosen from polyester, acrylic polymer and polyamide fibres.

6. Composition according to any one of claims 3 to 5, characterized in that the interferential fibres comprise a polymer chosen from the group consisting of polyethylene terephthalate, polyethylene naphthalate, polycarbonate, polymethyl methacrylate, nylon 6, nylon 6-6, nylon 6-12, nylon 11, nylon 12.

7. Composition according to any one of claims 3 to 6, characterized in that the interferential fibres are fibres with a multilayer structure of polymers comprising alternate layers of at least a first polymer and a second polymer.

8. Composition according to claim 7, characterized in that the said layers of polymers of the fibres are such that they allow the creation of a colour effect by interferences of light rays, which diffract and scatter differently according to the layers.

9. Composition according to claim 7 or 8, characterized in that each layer of polymer is in a plane (P) parallel to the direction of the principal axis of the fibre, in the direction of its length L.

10. Composition according to any one of claims 7 to 9, characterized in that the multilayer part of the fibre may comprise at least 5 individual layers of polymer, in particular from 5 to 120, preferably at least 10 layers, in particular from 10 to 70 layers, and better still from 10 to 50 layers.

11. Composition according to any one of claims 7 to 10, characterized in that each layer of the first and second polymers has respectively a thickness d₁, d₂ ranging, independently of each other, from 0.02 μm to 0.3 μm, and preferably from 0.05 μm to 0.15 μm.

12. Composition according to any one of claims 7 to 11, characterized in that the polymers present in the fibres have a refractive index ranging from 1.30 to 1.82 and better still ranging from 1.35 to 1.75.

13. Composition according to any one of claims 7 to 12, characterized in that the first and second polymers have respectively a refractive index n₁ and n₂ such that n₁/n₂ ranges from 1.1 to 1.4.

14. Composition according to any one of claims 7 to 13, characterized in that the fibre with multilayer structure has a reflection spectrum such that the width at half height of the spectrum λ_{L=1/2} is in the range 0<λ_{L=1/2}<200 nm.

15. Composition according to any one of claims 7 to 14, characterized in that the first polymer is a polyamide and the second polymer is a polyester.

16. Composition according to any one of claims 3 to 15, characterized in that the fibres are surface-treated or coated with a protective layer.

17. Composition according to claim 16, characterized in that the protective layer comprises a polymer chosen from the group consisting of polyurethanes, polyethyl acrylates, polyethyl methacrylates.

18. Composition according to claim 17, characterized in that the polymer of the protective layer has a refractive index ranging from 1.35 to 1.55.

19. Composition according to any one of claims 3 to 18, characterized in that the fibres have a length L and a diameter D such that L/D is chosen from the range going from 1.2 to 2500, preferably from 1.5 to 500, and better still from 1.6 to 150.

20. Composition according to claim 19, characterized in that the fibres have a section included in a circle having a diameter ranging from 2 nm to 500 μm, preferably ranging from 100 nm to 100 μm and better still from 1 μm to 70 μm.

21. Composition according to any one of claims 3 to 20, characterized in that the fibres have a length L ranging from 1 μm to 10 mm, preferably from 0.1 mm to 5 mm and better still from 0.3 mm to 3.5 mm.

22. Composition according to any one of claims 3 to 21, characterized in that the fibres are flat.

23. Composition according to any one of claims 3 to 22, characterized in that the fibres have a cross section having a longer length L1 and a shorter length L2 such that L1/L2 is greater than 4, preferably greater than 7.

24. Composition according to claim 23, characterized in that L1/L2 ranges from 4 to 15, preferably from 6 to 12, and better still from 7 to 10.

25. Composition according to any one of claims 22 to 24, characterized in that the flat fibres have a cross section of rectangular, ovoid or ellipsoid shape.

26. Composition according to any one of claims 3 to 25, characterized in that the flat fibres are provided in the form of ribbons or tagliatelle.

27. Composition according to any one of claims 3 to 26, characterized in that the fibres are monofilament or multifilament fibres.

28. Composition according to any one of claims 3 to 27, characterized in that the fibres are twisted along the axis of the length L of the fibres.

29. Composition according to any one of claims 3 to 28, characterized in that the fibres have a titre chosen from the range going from 0.15 to 30 denier and better still from 0.18 to 18 denier.

30. Composition according to claim 1, characterized in that the interferential particle is a pigment with interferential multilayer structure comprising at least two layers, each layer being made of at least one material chosen from the group consisting of the following materials: MgF₂, CeF₃, ZnS, ZnSe, Si, SiO₂, Ge, Te, Fe₂O₃, Pt, Va, Al₂O₃, MgO, Y₂O₃, S₂O₃, SiO, HfO₂, ZrO₂, CeO₂, Nb₂O₅, Ta₂O₅, TiO₂, Ag, Al, Au, Cu, Rb, Ti, Ta, W, Zn, MoS₂, cryolite, alloys, polymers and combinations thereof.

31. Composition according to claim 1, characterized in that the colouring agent is a pigment with interferential multilayer structure chosen from the group consisting of the structures:
Fe₂O₃/SiO₂/Fe₂O₃/SiO₂/Fe₂O₃;
MoS₂/SiO₂/mica-oxide/SiO₂/MoS₂;
Fe₂O₃/SiO₂/mica-oxide/SiO₂/Fe₂O₃.

32. Composition according to any one of the preceding claims, characterized in that the interferential particles are present in an amount ranging from 0.01% to 50% by weight, relative to the total weight of the composition, preferably from 0.1% to 30% by weight, and better still from 0.3% to 20% by weight.

33. Composition according to any one of the preceding claims, characterized in that the additional colouring agent is chosen from the group consisting of pigments, pearlescent agents, water-soluble or fat-soluble colorants, colouring polymers.

34. Composition according to any one of the preceding claims, characterized in that the additional colouring agent is a pigment chosen from titanium dioxide, zirconium oxides, cerium oxides, zinc oxides, iron oxides, chromium oxides, manganese violet, ultramarine blue, chromium hydrate, ferric blue, aluminium powder, copper powder, carbon black, D & C type pigments, and lacquers based on carmine, barium, strontium, calcium, aluminium.

35. Composition according to either of claims 33 and 34, characterized in that the pigments are present in an amount ranging from 0.01% to 15% by weight, relative to the weight of the composition, preferably from 0.01% to 15% by weight and better still from 0.02% to 5% by weight.

36. Composition according to any one of the preceding claims, characterized in that the colouring agent is a pearlescent agent chosen from mica coated with titanium or bismuth oxychloride, mica-titanium coated with iron oxides, mica-titanium coated with ferric blue or chromium oxide, mica-titanium coated with an organic pigment, pearlescent pigments based on bismuth oxychloride.

37. Composition according to one of claims 33 or 36, characterized in that the pearlescent agents are present in an amount ranging from 0.01% to 25% by weight, relative to the weight of the composition, preferably from 0.01% to 15% by weight, and better still from 0.02% to 5% by weight.

38. Composition according to any one of the preceding claims, characterized in that the colouring agent is chosen from water-soluble or fat-soluble colorants and colouring polymers, and is present in a colorant active substance content ranging from 0.01% to 6% by weight, relative to the total weight of the composition, preferably ranging from 0.01% to 3% by weight.

39. Composition according to any one of the preceding claims, characterized in that the interferential particles and the additional colouring agent are present in a weight ratio interferential particles/active substance of the additional colouring agent greater than or equal to 2, preferably ranging from 2 to 500.

40. Composition according to any one of the preceding claims, characterized in that the interferential particles and the additional colouring agent are present in a weight ratio interferential particles/active substance of the additional colouring agent greater than or equal to 5, preferably ranging from 5 to 500.

41. Composition according to any one of the preceding claims, characterized in that it comprises a hydrophilic or lipophilic cosmetic medium.

42. Composition according to any one of the preceding claims, characterized in that it comprises water or a mixture of water and hydrophilic organic solvent.

43. Composition according to any one of the preceding claims, characterized in that it comprises a fatty phase.

44. Composition according to claim 1, characterized in that the composition contains an ingredient chosen from oils, waxes, pasty fatty substances, gums, and mixtures thereof.

45. Composition according to any one of the preceding claims, characterized in that it contains an organic solvent.

46. Composition according to any one of the preceding claims, characterized in that it comprises a film-forming polymer.

47. Composition according to claim 45, characterized in that the film-forming polymer is chosen from the group consisting of vinyl polymers, polyurethanes, polyesters, polyamides, polyureas, cellulose polymers.

48. Composition according to either of claims 46 and 47, characterized in that the film-forming polymer is present in a polymer dry matter content ranging from 0.1% to 60% by weight relative to the total weight of the composition, preferably from 0.5% to 40% by weight, and better still from 1% to 30% by weight.

49. Composition according to any one of the preceding claims, characterized in that it comprises a cosmetic ingredient chosen from the group consisting of fillers, vitamins, thickeners, trace elements, emollients, sequestrants, perfumes, alkalinizing or acidifying agents, preservatives, or mixtures thereof.

50. Composition according to one of the preceding claims, characterized in that it is provided in the form of a nail varnish, mascara, eyeliner, hair composition, product for the lips, foundation, concealer, blusher or eyeshadow, product for the eyebrows, make-up product for the body.

51. Composition according to any one of the preceding claims, characterized in that it is provided in the form of a nail varnish.

52. Cosmetic method for applying make-up to the keratinous materials, characterized in that a composition according to any one of claims 1 to 51 is applied to the keratinous materials.

53. Cosmetic method for applying make-up to the keratinous materials comprising the application to the keratinous materials of a first layer, also called base layer, of a first cosmetic composition comprising, in a cosmetically acceptable medium, at least one colouring agent, then the application, to at least a portion of the said first layer, of a second layer of a second cosmetic composition in conformity with the composition as defined according to any one of claims 1 to 51, the first composition not comprising interferential particles as present in the second composition.

54. Method according to claim 53, characterized in that the first composition comprises a colouring agent chosen from pigments, pearlescent agents, water-soluble or fat-soluble colorants.

55. Method according to claim 53 or 54, characterized in that the first composition comprises a film-forming polymer.

56. Method according to any one of claims 53 to 55, characterized in that the first composition comprises a cosmetic ingredient chosen from the group consisting of fillers, vitamins, thickeners, trace elements, emollients, sequestrants, perfumes, alkalinizing or acidifying agents, preservatives, or mixtures thereof.

57. Method according to any one of claims 53 to 56, characterized in that the first composition is provided in the form of a nail varnish, mascara, eyeliner, hair composition, product for the lips, foundation, concealer, blusher or eyeshadow, product for the eyebrows, make-up product for the body.

58. Method according to any one of claims 53 to 57, characterized in that the first composition is provided in the form of a nail varnish.

59. Make-up kit comprising:
a first cosmetic composition comprising, in a cosmetically acceptable medium, a first colouring agent, and
a second cosmetic composition in accordance with the composition as defined according to any one of claims 1 to 51,
the first composition not comprising interferential particles as present in the second composition,
the first and second compositions being packaged in separate containers.

60. Make-up kit according to claim 59, characterized in that the first composition comprises a colouring agent chosen from pigments, pearlescent agents, water-soluble or fat-soluble colorants.

61. Make-up kit according to claim 59 or 60, characterized in that the first composition comprises a film-forming polymer.

62. Make-up kit according to any one of claims 59 to 61, characterized in that the first composition comprises a cosmetic ingredient chosen from the group consisting of fillers, vitamins, thickeners, trace elements, emollients, sequestrants, perfumes, alkalinizing or acidifying agents, preservatives, or mixtures thereof.

63. Make-up kit according to any one of claims 59 to 62, characterized in that the first composition is provided in the form of a nail varnish, mascara, eyeliner, hair composition, product for the lips, foundation, concealer, blusher or eyeshadow, product for the eyebrows, make-up product for the body.

64. Make-up kit according to any one of claims 59 to 63, characterized in that the first composition is provided in the form of a nail varnish.

65. Support to which make-up has been applied comprising a make-up which is capable of being obtained according to the make-up application method in accordance with any one of claims 52 to 58 and applied to the said support, the said support being chosen from false nails, false eyelashes, pastiches, wigs, pastilles or patches adhering to the skin or the lips.
